(19) United States
(12) Patent Application Publication
(10) Pub. No.: US 2013/0273658 A1
(43) Pub. Date: Oct. 17, 2013

(54) HUMAN ADIPOCYTE-DERIVED BASEMENT MEMBRANE FOR TISSUE ENGINEERING APPLICATIONS

(71) Applicant: WILLIAM MARSH RICE UNIVERSITY, Houston, TX (US)

(72) Inventors: Aaron Damm, Houston, TX (US); Deepak Nagrath, Houston, TX (US)

(73) Assignee: WILLIAM MARSH RICE UNIVERSITY, Houston, TX (US)

(21) Appl. No.: 13/861,105

(22) Filed: Apr. 11, 2013

Related U.S. Application Data
(60) Provisional application No. 61/622,577, filed on Apr. 11, 2012.

(51) Int. Cl. C12N 5/071

(52) U.S. Cl. CPC C12N 5/0653; C12N 5/0676
USPC 435/377

(57) ABSTRACT

The present disclosure generally relates to tissue engineering. More particularly, the present disclosure relates to the generation of a new biologically active and biocompatible human derived materials useful in tissue engineering applications.

### BACKGROUND

Tissue and organ replacement has been limited to either transplantation with donor tissue or more recently, replacement with synthetic biomedical devices. However, both strategies suffer from significant disadvantages. Transplantation is limited by the number of healthy organ donors as well as viability of donated organs. While biomedical devices do not suffer from limited number, they lack the same self renewal properties of native tissue. To combat these shortages, researchers have taken up the field of tissue engineering. Tissue engineering was defined by Langer et al. as an interdisciplinary field that applies the principles of engineering and life sciences toward the development of biological substitutes that restore, maintain, or improve tissue function.

Current tissue engineering strategies focus on replicating the native extracellular matrix (ECM) through either synthetic or natural, biologically-derived, scaffolds composed of engineered polymers or proteins. Synthetic scaffolds offer the promise of cost-effectiveness and reproducibility with defined mechanical characteristics as well as degradation in vivo. While synthetic scaffolds have shown promising results and advanced designs, these materials also introduce foreign agents into the body and have difficulty in mimicking the complexity of the native extracellular matrix. Biologically-derived extracellular matrix increases the complexity of the engineered scaffold, but suffers from undefined composition, difficulty in reproduction, and lack of donor tissue.

Recently, researchers have begun investigating the use of natural extracellular matrix deposits on synthetic scaffolds. A study done by Chen et al investigated the effects of human mesenchymal stem cells (hMSCs), fibroblast and chondrocyte cultured cell-derived extracellular matrices on hMSCs and fibroblasts. To produce the cell-derived matrices, either fibroblasts, hMSCs or chondrocytes were grown on polymer scaffolds for an extended period of time, and then each construct was decellularized. The remaining construct contained the secreted ECM and was re-seeded with hMSCs or fibroblasts. While this study did not compare hMSC-derived scaffolds with fibroblast-derived scaffolds directly, both constructs showed higher proliferation, bioactivity and filled in the voids in the construct with their own ECM after re-seeding with the same cell type. The researchers were able to show that the natural cell-derived ECM is a viable option for improving polymer scaffolds. They also kindled the idea of using different cell types for specific applications.

Rat tail collagen and Matrigel are popular reconstituted ECMs currently used for in vivo 3D tissue cultures and are models for reconstituted cell-secreted ECM. A study done by Leach et al cultured human mesenchymal stem cells on tissue plastic to generate a matrix for accelerating the osteogenic differentiation of hMSCs. The cell-secreted matrix was collected from the original well after decellularization, purified and re-seeded on another cell culture plate for use with hMSCs. While the main goal of the study was to accelerate differentiation, the ability to transfer the matrix to another well shows the viability of cell-secreted matrices as reconstituted ECM. These soft gels have the ability to adapt to nearly any geometry while maintaining structural integrity. Researchers at Harvard University have demonstrated the ability to create a cell-secreted ECM gel similar to collagen from 3T3-L1 mouse preadipocytes. This mammalian matrix demonstrated the ability to improve rat hepatocyte function in 3D cultures when compared to the current standard collagen sandwich culture. When a drop of this concentrated ECM was placed on a petri dish, the ECM held the droplet shape and did not disperse, demonstrating its potential as a soft moldable scaffold.

### DRAWINGS

Some specific example embodiments of the disclosure may be understood by referring, in part, to the following description and the accompanying drawings.

FIG. 1 shows the experimental design for adipose-derived ECM generation.

FIG. 2 shows bright field images of adipocyte differentiation over a 16 day treatment period.

FIG. 3 shows Oil Red O staining of normally differentiated adipocytes over a 16 day period.

FIG. 4 shows SGBS secreted protein before concentration with statistics (* P≦0.05, ** P≦0.01, ***P≦0.001). Control condition was fresh unexposed media.

FIG. 5 shows concentrated protein content for SGBS cells during differentiation induction. The control was filtered unexposed media.

FIG. 6 shows Western blot of Laminin in concentrated adipocyte secreted ECM.

FIG. 7 shows humagel viability experimental design.

FIG. 8 shows stimulation index, Trial 1 of Islets Seeded in Single, Double and Embedded Collagen and Humagel overlay.

FIG. 9 shows Insulin Content, Trial 1 of Islets Seeded in Single, Double and Embedded Collagen and Humagel Overlay.

FIG. 10 shows Stimulation Index, Trial 2 of Islets Seeded in Single, Double and Embedded Collagen as well as Humagel (*P≦0.05, ** P≦0.01, *** P≦0.001).

FIG. 11 shows Insulin Content, Trial 2 of Islets Seeded in Single, Double and Embedded Collagen as well as Humagel (*P≦0.05, ** P≦0.01, *** P≦0.001).

FIG. 12 shows Stimulation Index, Trial 3 of Islets Seeded in Single, Double and Embedded Collagen as well as Humagel overlay and Humagel/Collagen Embedded (*P≦0.05, ** P≦0.01, *** P≦0.001).

FIG. 13 shows Insulin Content Trial 3 of Islets Seeded in Single, Double and Embedded Collagen as well as Humagel overlay and Humagel/Collagen Embedded (*P≦0.05, ** P≦0.01, *** P≦0.001).

FIG. 14 shows Islet morphology on days 7 and 14 in culture.

FIG. 15 shows Bright Field Imaging of Differentiated Adipocytes.

FIG. 16 shows Oil Red O Staining for Adipogenesis Induction Potential.

FIG. 17 shows a schematic for metabolic engineering of preadipocytes to produce ECM used for islet or soft tissue.

FIG. 18 shows Bright Field Imaging and Oil Red Staining for Differentiation where AA1 is proline supplement, AA2 is hydroxyproline, L is linoleic acid, O is oleic, P is palmitic, RO is rosiglitazone, and Ost is osteocalcin.

FIG. 19 shows Protein Content for SGBS Stimuli During Differentiation where AA1 is proline supplement, AA2 is hydroxyproline, L is linoleic acid, O is oleic, P is palmitic, RO is rosiglitazone, and Ost is osteocalcin.

FIG. 20 shows Percent Change in Protein Content Compared to Normal Differentiation where AA1 is proline supplement, AA2 is hydroxyproline, L is linoleic acid, O is oleic, P is palmitic, RO is rosiglitazone, and Ost is osteocalcin.

FIG. 21 shows Protein Content on Day 6 with Statistical Analysis (* P≦0.05) where AA1 is proline supplement, AA2 is hydroxyproline, L is linoleic acid, 0 is oleic, P is palmitic, RO is rosiglitazone, and Ost is osteocalcin.

FIG. 22 shows Protein Content on Day 8 with Statistical Analysis (* P≦0.05, ** P≦0.01, *** P≦0.001) where AA1 is proline supplement, AA2 is hydroxyproline, L is linoleic acid, O is oleic, P is palmitic, RO is rosiglitazone, and Ost is osteocalcin

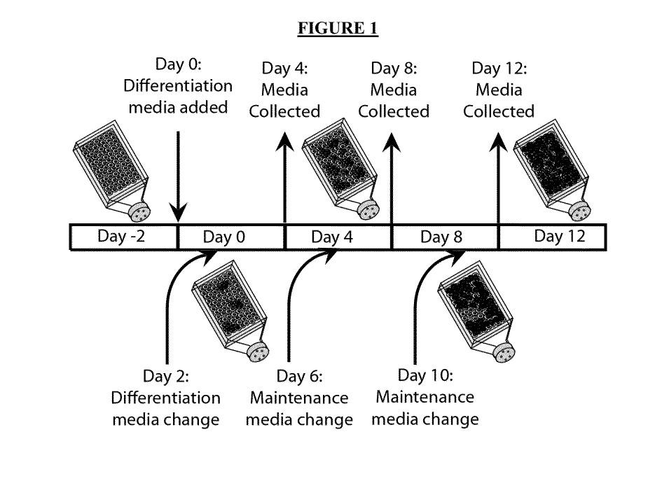

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to tissue engineering. More particularly, the present disclosure relates to the generation of a new biologically active and biocompatible human derived materials useful in tissue engineering applications.

Current tissue engineering strategies focus on replicating the native extracellular matrix (ECM) through either synthetic or natural, biologically-derived, scaffolds composed of engineered polymers or proteins. Synthetic scaffolds have evolved from basic building blocks into complex polymer scaffolds chemically constructed to promote cell adhesion. Nearly all synthetic scaffolds are derived from a small group of materials originally used as degradable suture material. The most commonly used materials are poly(glycolic acid) (PGA) and poly(lactic acid) (PLA) from the poly(α-hydroxyesters). These scaffolds offer the promise of cost-effectiveness and reproducibility with defined mechanical characteristics as well as degradation in vivo. While synthetic scaffolds have shown promising results and advanced designs, these materials also introduce foreign agents into the body and have difficulty in mimicking the complexity of the native extracellular matrix.

Numerous methods have been developed for isolating natural ECM from a variety of sources, primarily using a decellularization process. One of the most promising methods is the use of sacrificial open celled foams for in vitro expansion of ECM secreting cells. In this method, the cells are removed from the foam leaving behind the synthesized ECM, then the foam is chemically degraded leaving behind only the secreted ECM. A similar method uses progenitor cells to deposit a native extracellular matrix on a titanium scaffold to enhance biological activity of the scaffold. The progenitor cells are removed from the scaffold after depositing an ECM layer on the scaffold, and the scaffold is reseeded with marrow stromal cells to be differentiated in to osteoblastic cells. Both methods have shown promising results for producing ECM and enhancing the effectiveness of current scaffolds. However, these methods are still dependent on donor tissue (decelluarized organs) and enzymatic or chemical procedures for isolation of the ECM. Biological ECM derived from animals is also limited by the structure and composition of the harvested tissue, because it does not perfectly match the characteristics of human ECM. Nevertheless, the use of cells to deposit ECM in vitro for use as a scaffold has lead to the development of cell synthesized ECM processes.

Another commercially viable source of human ECM proteins is through genetic engineering. Researchers have successfully produced human collagen from bacteria, yeast, Chinese ovarian hamster cells, and HeLa cells. Since in vivo collagen synthesis is an intricate process and essential for cell survival, recombinant collagen is unable to identically match the native collagen conformation. However, methods using recombinant collagen have been used to create scaffolds for skin and bone engineering applications. The combination of genetic engineering and cell deposited ECM processes has culminated in the development of human cell secreted ECM capable of modulation to produce designer specific scaffolds.

Recently, bioengineers have produced a new type of biological ECM. This is cell-secreted extracellular matrix (CS-ECM), an ideal biomaterial for tissue engineering. CS-ECM is produced by cells in culture and can either be used as a reconstituted ECM or as a decellularized construct. Native tissue consists of a variety of cell types and the extracellular matrix secreted by them. Native extracellular matrix is known to be involved in cell signaling, mobility, and proliferation. This matrix provides the ideal environment for cell growth and adhesion. ECM deposited by cells in vitro can be collected to produce human derived biologically active materials. Okano pioneered a method of cell sheet engineering using temperature sensitive poly(N-isopropylacrilamide) (PI-PAAm). When the polymer surface is cooled below 32° C., the surface is becomes hydrophilic, resulting in spontaneous cell detachment. The detached sheets contain viable cells, intact cell to cell junction proteins, growth factor receptors, as well as deposited ECM. Okano also demonstrated the usefulness of this approach for islet transplantation. The islet cell sheets were then transplanted and maintained their functionality for 7 days in vivo. However, the thickness of the sheets makes them a less viable method for larger tissues even though they can be stacked together.

CS-ECM may hold the key to overcoming current obstacles in scaffold development. CS-ECM can be obtained from cultured cells, thus overcoming the need for donor tissue. Also, several secretome profiling studies suggest that the composition of the secreted extracellular matrix is defined depending on the type of cell used. Cell-secreted ECM has shown promising results for numerous applications, from articular cartilage repair to bone replacement. Many of the advantages that cell-secreted ECM offers have yet to be fully developed or found. At the forefront of these advantages is the ability to metabolically engineer human cells to produce large quantities of a targeted tissue-specific ECM. For example, long-term culture in ascorbic acid has been shown to promote collagen synthesis in skin fibroblasts. Another study by Mikos et al. uses shear stress to induce ECM generation by rat marrow stromal cells. These studies suggest that, depending on the culture conditions and substrates available, mammalian cells can be manipulated to produce desired protein products. Another advantage of cell-secreted ECM is that pathogens can be identified and removed in vitro, preventing host inflammatory response. Finally, CS-ECM inherently possesses the complexity of the native extracellular matrix required for cell signaling, cell repopulation, and ECM remodeling. These advantages have lead researchers to study the ECM generated by numerous cell types and sources.

CS-ECM is a promising new material for tissue engineering applications, offering a naturally-derived material capable of mimicking the complexity of native ECM, while avoiding eliciting foreign body inflammatory responses from the host. Although most recent studies demonstrate the use of cell-secreted ECM from various cell sources as producing favorable results for enhancing cell proliferation and function on decellularized polymer scaffolds, there remains the ability to use the ECM as reconstituted scaffold similar to matrigel, capable of taking different geometries based on application constraints. Future work with cell-secreted ECM also elicits ideas of metabolically engineering mammalian cells for production of large quantities of ECM containing high concentrations of vital growth factors. While the use of multiple cell types that can be co-cultured and metabolically manipulated to produce a biomaterial that resembles the native structure of the desired tissue has yet to be established, cell-secreted ECM holds the promise of producing a biomaterial for tissue engineering.

The present disclosure provides for the development and engineering of a scaffold comprising material naturally secreted by human preadipocytes during differentiation. During this differentiation process, the preadipocytes remodel the extracellular matrix by releasing new extracellular proteins. Finally, the viability of the new basement membrane as a scaffold for tissue engineering using human pancreatic islets, and as a scaffold for soft tissue repair, was investigated. After identifying the original scaffold material, improvement of the yield of material was sought by treating the cell as a bioreactor, through various nutritional and cytokine stimuli. The present disclosure suggests that adipocytes can be used as bioreactors to produce a designer-specified engineered human extracellular matrix scaffold for specific tissue engineering applications.

With the goal of generating a natural human-derived ECM scaffold safe for human transplantation, in certain embodiments of the present disclosure, preadipocytes were used because of the abundance of subcutaneous fat in the body and previous success with mouse preadipocytes. In certain embodiments, SGBS preadipocytes may be in conjunction with the methods of the present disclosure due to their recognition as a model for human subcutaneous preadipocytes and their primary cell lineage.

The extracellular matrix is a complex and intricate material that provides structural and functional support comprised of glycoproteins, fibronectin, collagens, laminins, and growth factors. The exact composition of the extracellular matrix varies based on cell phenotype and function. Due to the complexity of natural ECM, a natural in vitro cell culture system capable of generating sufficient amounts of ECM with growth factors has to be established. As previously mentioned, numerous cell types produce natural ECM, but they do not produce sufficient material. The present disclosure provides for a novel human preadipocyte differentiation system capable of generating substantial amounts of ECM.

To attempt to overcome the shortcomings, including immune response and lack of donors, of current tissue engineering scaffolds, the present disclosure provides for the use human adipocytes to generate a material capable of serving as a tissue engineering material free of pathogens. The technique developed uses the differentiation of preadipocytes as the primary source of extracellular matrix proteins. During the differentiation from preadipocytes to adipocytes, the morphology of the cells changes. As the cells differentiate, to compensate for the changing morphology, they produce ECM proteins to remodel their extracellular matrix surroundings. The technique collects the secreted ECM proteins and filters them for use as a tissue engineering scaffold. To enhance the production of ECM proteins during differentiation, different supplements are used to stimulate ECM production. The present disclosure lays the framework for developing a method for producing a human adipocyte-derived extracellular matrix material with defined composition that is capable of being manipulated for varying tissue engineering applications.

One of the most daunting challenges faced in tissue engineering is finding a safe, abundant source of scaffolding material. While polymer-based scaffolds are naturally derived, studies have shown they still incite foreign body responses. Animal tissues have served as an alternative to synthetic scaffolds, yet they lack the same biological composition as the human extracellular matrix. The material of the present disclosure offers several advantages over current scaffold systems. Due to the culture conditions, the material should be pathogen-free and is derived from human preadipocytes thus, as shown in another study, should not elicit a host immune response. Also, due to the human cell origin, the material closely resembles endogenous human tissue. Finally, this technique can completely eliminate any host response by using patient-derived cells to generate the scaffolding. Although the production of material from the patient-derived adipocytes would require an additional harvesting operation with an extended culture period, the resulting material could be coupled with patient-derived stem cells to create an individually-matched cell therapy for damaged tissue.

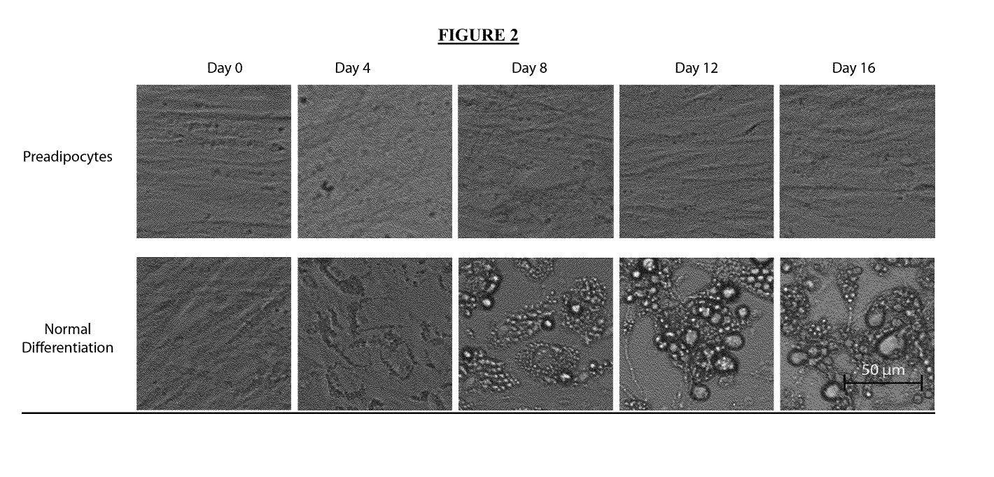

In certain embodiments, Simpson-Golabi-Behmel Syndrome (SGBS) preadipocytes may be used to generate the materials of the present disclosure. When confluent Simpson-Golabi-Behmel Syndrome (SGBS) preadipocytes were cultured in the serum free DMEM/F12 media without adipogenic factors, the cells did not undergo differentiation, indicating there is no self-differentiation potential. When adipogenic factors (insulin, cortisol, triiodothyronine, rosiglitazone, dexamethasone, and IBMX) were introduced to confluent SGBS preadipocytes in serum free DMEM/F12, the majority of SGBS cells differentiated into mature adipocytes as seen in FIG. 2. During the differentiation period, the cells accumulated lipids and displayed a round shape with a cytoplasm full of lipid droplets which are visual markers for fully differentiated adipose cells. Oil Red O staining confirmed the presence of lipid droplets in the cytoplasm of the treated cells, which is consistent with previously reported data by Wabitsch et al.

When the cell-exposed media was collected, there was a noticeable difference in consistency between the fresh media and exposed media. Rosenow et al previously studied the secretome of SGBS cells pre- and post-differentiation and identified numerous ECM proteins and growth factors in the secretome but did not quantify the protein secretion levels. Our results are consistent with these findings. Protein secretion levels were significantly elevated during differentiation, and western blotting confirmed the presence of laminin, an extracellular protein, in the concentrated media. However, the concentrated and unconcentrated forms of material revealed different trends in protein secretion. The unconcentrated protein content was highest on Day 2 and continued to decrease over the course of differentiation. The concentrated protein content had a bell-shaped trend with the highest protein content on Day 8, followed by Day 4 and the lowest on Day 12. Since a 100 kDA filter was used for concentration, this trend suggests that large proteins are secreted primarily on Day 8 while smaller proteins are secreted earlier in the differentiation process on Days 2 and 4. Since the smaller proteins are likely growth factors and signaling molecules, for certain applications a smaller filter size could be used on the filtrate to collect these proteins and combine them with the larger ECM proteins for an ideal scaffold.

Thus, during differentiation, SGBS cells secrete a protein mixture consisting of ECM proteins as well as growth factors identified in previous studies of adipocyte differentiation. According to the present disclosure, the secreted protein mixture can be concentrated for use as a human-derived ECM scaffold for tissue engineering applications. As used in the present disclosure, this concentrated protein mixture will be referred to as humagel.

Islet Tissue Engineering Using Humagel Scaffolds

Islet tissue engineering and regenerative medicine is one of the most promising and challenging fields for treating type 1 diabetes. The World Health Organization estimates 346 million people currently suffer from diabetes, and that 366 million people worldwide, nearly 4.4% of the total population of Earth, will suffer from diabetes by 2030. Islet transplantation for type 1 diabetes patients offers the potential to stabilize glucose metabolism and prevent further diabetic complications. However, islets from more than one donor are generally required to achieve euglycemia, and transplant patients are still affected by reduced glucose-stimulated insulin secretion. Furthermore, the majority of these treatments provide insulin independence for only five years. While the causes of graft failure are not completely known, the disruption of the basement membrane and the vasculature structure are likely culprits. During isolation, the islets are enzymatically cleaved from the basement membrane, destabilizing the islets and disrupting the vascular connections. The basement membrane (BM) provides structural support, mediates adhesion, and activates chemical signaling pathways within the cells. When the islets are removed from the BM, they undergo apoptosis. Repairing the BM connections through the use of ECM components in islet cultures has been shown to increase islet survival and functionality. More detailed studies have shown the exact effect of different ECM proteins on the functionality of islets. These studies found a direct correlation between Collagen I, Collagen IV, and islet adhesion and glucose metabolism as well as fibronectin and islet morphology and insulin release. The present disclosure suggests that there exists an optimal concentration of each ECM component for preserving islets in vitro. Researchers have also shown the usefulness of polymer scaffolds coupled with various ECM components. The scaffold was coupled with a combination of ECM components to preserve islet functionality in long term cultures.

Another important aspect of the native in vivo islet environment is the various growth factors constantly interacting with the islets, the most important of which is vascular endothelial growth factor (VEGF). Studies have shown that the inclusion of VEGF in islet media increased the functionality and survivability of islets over time. All of these studies suggest that maintaining islet function in culture for transplantation will require the incorporation of ECM components, growth factors and nutrients.

The new BM derived from murine adipocytes consists of ECM components that have been shown to have a positive effect on islets; these include fibronectin, collagen IV, hyaluronan, and laminin as well as various growth factors that promote islet function and viability such as vascular endothelial growth factor (VEGF) and adiponectin. Furthermore, work on human preadipocytes has shown that during differentiation to mature adipocytes, the preadipocytes secrete various ECM and growth factors as well. The present disclosure investigated the effectiveness of naturally-derived human basement membrane extract (BME) for long term in vitro culture of human pancreatic islets. The effect of the produced BME on islet function was determined by the glucose-stimulated insulin response test and calculation of the stimulation index. Insulin content was also measured to further demonstrate the islet function.

To improve the success rate of islet transplantation, the effect of the humagel of the present disclosure on human pancreatic islets was investigated. While the main cause of failures in islet grafts has not been identified, a leading candidate is the disruption of the islet ECM environment resulting in the loss of islet function and islet cell death. The functionality of islets in culture is also linked to their morphology. Since the humagel is composed of human ECM isolated from adipocytes, the humagel could be able to mimic the islet ECM microenvironment.

Examination of the islet morphology revealed significant changes in boundaries of the islets. In the single collagen gel, the islets began to spread out from the original islet boundary with a web-like appearance, while the embedded and double gel displayed little or no change in their boundaries, consistent with previous studies. However, the humagel condition also experienced significant spreading from the original boundaries. Since there is a second layer of ECM over the islets, they were not expected to spread out and were expected to behave similarly to the double gel conditions. The spreading effect may be due to adding humagel in small quantities over the course of several days instead of a large amount of humagel at once to form a complete second layer of ECM construct over the islets. However, when the humagel islets were stained (data not shown) for insulin, the cells branching out from the islet boundaries stained positive for insulin. This suggests that the cells breaking out of the islet are β-cells that have either migrated from the islet or are newly formed β-cells. While β-cell proliferation was not thought to occur previously, there is recent evidence supporting β-cell proliferation in rare instances.

For the first trial, the glucose challenge test clearly indicated that islets cultured with humagel had a higher functionality than islets cultured only in the presence of collagen. This result prompted a more comprehensive study of the effect of humagel on islet function in vivo. The second trial demonstrated the same increased SI in humagel as compared to the other conditions. The increased stimulation index suggests that the combination of ECM proteins in the humagel provides a favorable environment over only collagen IV. The suggestion that a mixture of ECM proteins would more closely mimic the native islet environment is supported by recent evidence that each ECM protein is responsible for different interactions with islets, with a mixture of ECM proteins resulting in the optimal environment for islet function. The higher functionality of the islets in humagel was further confirmed by insulin content measurements, where the humagel had the highest insulin content except for the double gel. This suggests that not only does the humagel increase the islet stimulation index, but it also increases the islet glucose sensitivity.

However, the increased stimulation index was lower than expected values when compared to previously reported data for ECM effect on islet function. Furthermore, based on the morphology of the embedded collagen islets and previous studies supporting the use of three dimensional culture conditions, a new condition embedding islets in a mixture of collagen and humagel at a 3:2 ratio was proposed. For the third trial, the digestion procedure was modified for glucose challenge testing from mechanical digestion to collagenase digestion. With this change, the stimulation index of the humagel cultured islets decreased significantly. It is well known that islets exposed to collagenase for extend periods of time begin to break down. A possible explanation is that the collagenase damaged the islets in the humagel because of the decreased collagen content in the gel when compared to the other collagen preparations.

Humagel in Soft Tissue Applications

Since current autograft procedures primarily inject isolated fat from other sections of the body into the defect, they are unsatisfactory for long-term replacement due to poor vascularization and poor adipocyte viability, similar to the factors plaguing islet transplantation. Tissue engineering strategies offer the promise of an ideal adipose tissue substitute capable of predictable soft tissue volume and shape as well as a high degree of vascularization. Recent adipose tissue engineering strategies have focused on scaffolds seeded with adipocyte precursors; of particular interest is the use of naturally derived collagen, fibrin, and hyaluronan. The discovery of adipose tissue as a source of adult multipotent stem cells has sparked interest in adipose tissue engineering. The use of scaffolds seeded with these adipose-derived adult multipotent stem cells has generated promising results for soft tissue reconstruction. However, as with any tissue engineering strategy, the scaffold must also be biodegradable and avoid immune response. Since few models have reached the clinical realm, there remains a need to engineer a suitable adipose tissue substitute for reconstructive or plastic surgery.

According to the present disclosure, the newly formed humagel was investigated for use as a scaffold for adipose tissue substitutes in soft tissue defects. The humagel makes use of its adipose origin and the abundance of extracellular matrix proteins secreted during the differentiation process to provide an ideal scaffold for adipose tissue regeneration. To investigate the potential of humagel as a scaffold for adipose tissue regeneration, the in vitro adipogenic potential was compared with that of a normal differentiation procedure as well as the necessary control groups.

There is a growing need for a suitable adipose tissue substitute capable of filling a defined shape and volume for reconstructive purposes. Here, the use of humagel as an adipogenesis agonist has been demonstrated. The untreated preadipocytes did not accumulate lipid droplets as shown in the bright field images, or stain with Oil Red O for lipid droplets, indicating that there is not a self-induced differentiation present in the cells. The normal and rosiglitazone treated preadipocytes both developed into mature adipocytes as seen by the Oil Red O staining of lipid droplets in the cytoplasm as well as their rounded morphology seen in bright field imaging. The humagel also induced differentiation in preadipocytes, although in a different form. The preadipocytes exposed to humagel started to show small lipid droplets on Day 4 and the droplets continued to increase in number as seen on Day 8. On Days 12 and 16, some of the lipid droplets in the humagel-treated adipocytes were similar in size to those in the normal differentiation group, but the majority of droplets were similar to the small droplets seen on Days 4 and 8. The size of these lipid droplets suggest that adipocytes differentiated using humagel are more insulin-sensitive than the normal or rosiglitazone differentiated adipocytes. Thus, humagel could be used for differentiating a control group of adipocytes for studying insulin resistance.

However, a far more significant result is the differentiation potential of humagel and its potential use as a scaffold for adipose tissue regeneration. The effectiveness of humagel for inducing preadipocyte differentiation to healthy mature adipocytes is the first step in the development of humagel as a scaffold for soft tissue repair. The inherent advantages of humagel as a scaffold, coupled with its adipogenic induction potential, is a promising start for soft tissue repair.

Bioreactor for Designer Tissue Engineering Scaffolds

According to the present disclosure, the concentrated material generated during differentiation may lack sufficient protein concentrations to be useful for some tissue engineering applications. For more strenuous applications such as bone tissue engineering, an ideal scaffold would have an increased quantity of structural proteins such as collagen as compared with scaffold engineering. To increase the effectiveness of human cells as bioreactors for producing tissue engineering scaffolds, techniques for optimizing the production of ECM proteins were investigated.

The effect of mechanical stimuli on cell ECM production is well studied for bone tissue engineering applications. A study on osteoblasts cultured in perfusion chambers investigated the effect of fluid flow on mineralized matrix deposition. By exposing the osteoblasts to varying rates of flow for 16 days, they were able to increase the amount of matrix deposited on the scaffold as compared to normal static cultures. Numerous other studies have studied the effect of mechanical stimuli on cultured cell ECM production. Of the many reactions to mechanical stimuli, the most strategic for tissue engineering applications were the alignment and improved production of ECM proteins. During preliminary testing, adipocytes failed to maintain attachment in under flow conditions and were washed away. A second proposed method for mechanically induced ECM production was to place the cells in a slightly stressed environment. This method is founded on the idea that cells will react through signaling pathways to stresses to return to homeostasis with their environment. Based on this idea, according to the present disclosure, trypsin, dispase and collagenase was used to cleave the cells from their ECM in vitro, forcing them to generate more ECM to return to homeostasis.

In other embodiments, cell ECM production is modified through hormonal and nutritional stimulation. Recent research has discovered that bones play a crucial part in global energy metabolism and adipocyte secretion of adipokines Osteoblast-specific secreted osteocalcin interacts with both β-cells and adipocytes, participating in the regulating global energy metabolism. Recently published data indicates that osteocalcin effects adiponectin secretion in adipocytes. While this demonstrated osteocalcin's effect on ECM protein secretion by mature adipocytes, we investigated a similar effect on differentiating preadipocytes. Extensive research on enhancing collagen and proteoglycan synthesis in skin fibroblasts has shown that long term exposure to specific amino acids can increase the production of collagen and proteoglycans. While these studies focused on the use of topical agents to reduce the signs of aging, they suggest that amino acid additives such as ascorbic acid, proline, glycine and lysine could induce fibroblasts to increase extracellular matrix production. Even though these studies were on skin fibroblasts, the basic principles reflect the regulation of collagen synthesis and thus should be transferable to all cell types that generate collagen, including adipocytes. Similar experiments have demonstrated the effect of linoleic acid, oleic acid, and palmitic acid on adipocyte differentiation and lipid accumulation. Adipocytes have the unique capacity to store excess free fatty acids as triglyceride in lipid droplets, thus it was expected that increased lipid accumulation in adipocytes possibly leading to increased extracellular matrix remodeling. For these reasons it was hypothesized that by different nutritional supplements, the amount and composition of the secreted basement membrane could be enhanced and manipulated for varying applications.

One of the main challenges of tissue replacement therapies is the ability to generate biological material within cell seeded supports after transplantation. To decrease the amount of initial time and material needed for cell seeded scaffolds to successfully attach to the damaged area, it is desirable for the cells to produce their own ECM as well as use the starting material provided by the scaffold. Based on published evidence indicating that certain nutritional supplements can increase the ECM production in skin cells when applied topically, it was hypothesized that the same nutritional and hormonal supplements could increase ECM production in human preadipocytes in vitro during differentiation, effectively using the preadipocytes as a bioreactor to produce ECM based on the effects of different media supplements.

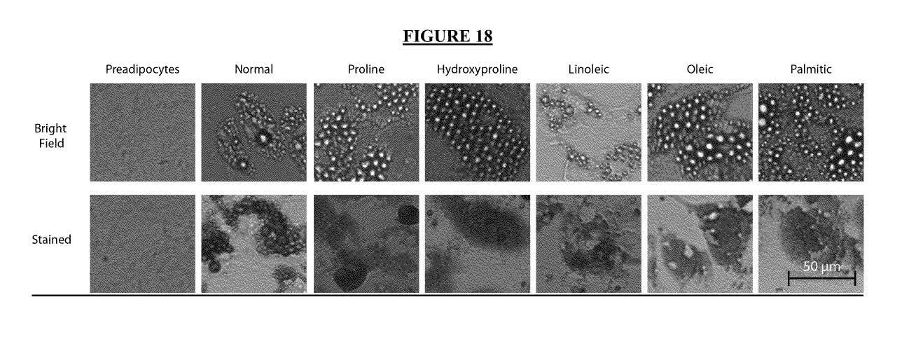

The protein content results indicate that linoleic acid, palmitic acid and proline are all capable of increasing the yield of ECM within the collected media, although palmitic acid- increased secretion was not statistically significant. Linoleic acid was the only supplement to increase protein secretion over the whole differentiation process and has not been previously reported. Prior studies have shown that linoleic acid promotes lipid filling of murine adipocytes while inhibiting proliferation. The increased lipid accumulation is also seen in this study in FIG. 18. A possible explanation for increased protein secretion is that the increased lipid accumulation required an increase in extracellular matrix remodeling and ultimately produced more proteins for the process. Oleic acid also demonstrated an increase protein secretion on Day 8 and increased lipid accumulation. While the increased protein secretion was only 5% higher than the normal-differentiated group—the increased lipid accumulation was expected from previous studies. Palmitic acid-treated adipocytes also demonstrated a high degree of lipid accumulation, an expected result when compared with previous studies on adipocytes. The unique capacity of adipocytes for storing excess free fatty acids in lipid droplets as triglyceride was demonstrated in the fatty acid-treated conditions, along with increased protein secretion seen in the palmitic and lanoleic cases.

The increased protein content in the proline condition was consistent with the published results in skin fibroblasts, demonstrating that proline supplementation does increase ECM production. The protein content in the proline-treated adipocytes was 10% greater on Day 8. Hydroxyproline was not expected to produce an increase in protein secretion, because only the hydroxylation of proline and lysine by proline hydroxylase and lysine hydroxylase allows collagen synthesis to proceed in vivo. Rosiglitazone and osteocalcin both produced a decrease in protein secretion. However, these conditions detached from the culture plates during the second experiment. During the first trial, they produced levels of protein during differentiation similar to the normal differentiation. While the secreted protein was not quantitatively characterized, the ability to increase or decrease protein secretion by preadipocytes during differentiation demonstrates the usefulness of cells as a bioreactor for engineering designer tissue engineering scaffolds based on application constraints.

While the methods of the present disclosure suggest a patient-matched method for overcoming immune response, the additional operation and extended culture period are drawbacks of this method. Future work in this area should revolve around developing an autologous extracellular matrix scaffold capable of use in all patients and free of the extended culture period. Research into the creation of an autologous matrix has already begun. Researchers at the University of Tsukuba developed a material by decellularizing a template seeded with autologous cells. After removing the template, the remaining ECM demonstrated a high degree of biocompatibility. While this method still exposes the matrix to exogenous materials and requires further development, they demonstrated the potential development of an autologous extracellular matrix. Future directions should also include mapping the precise relationship between stimulants and the secreted ECM. While the precise conditions for producing a specific product will vary by cell type, the ability to enhance cell-secreted matrix should be further developed. Manipulating the cell's secretome would naturally progress towards using cells as bioreactors to meet design criteria for assorted applications. Another part of the cell as a bioreactor concept is the use of different cell types in co-culture to produce a biomaterial that more closely mimics the native environment of the desired tissue.

Cell-secreted extracellular matrix is a promising new material for tissue engineering applications, offering a naturally-derived material capable of mimicking the complexity of native ECM while avoiding eliciting foreign body inflammatory response from the host. Although most recent studies demonstrate the use of cell-secreted ECM from various cell sources as producing favorable results for enhancing cell proliferation and function on decellularized polymer scaffolds, there remains the ability to use the ECM as reconstituted scaffold similar to matrigel, capable of taking different geometries based on application constraints. Future work with cell-secreted ECM also elicits ideas of metabolically engineering mammalian cells for production of large quantities of ECM containing high concentrations of vital growth factors. While the use of multiple cell types in co-culture and metabolically manipulating cells to produce a biomaterial that resembles the native structure of the desired tissue has yet to be established, cell-secreted ECM holds the promise of producing an ideal biomaterial for tissue engineering.

In certain embodiments, humagel may be used as a scaffold for encapsulating islets for transplantation. Research has shown that islets cultured in 3-dimensional structures have improved function and viability for transplantation. The developed basement membrane would duplicate the peri-insular basement membrane and cell-cell interactions in the islets increasing islet survival. Another challenge in islet engineering and transplantation is the lack of vascularization. A basement membrane in mice has been shown to promote angiogenesis and endothelial cells co-cultured with adipocytes have been shown to have increased tube formation. These results suggest that humagel would promote vascularization and survival in isolated and transplanted islets as well as possibly promoting β-cell proliferation. An even more intriguing aspect of humagel is the potential to stimulate β-cell proliferation in vitro. If humagel is promoting β-cell proliferation, β-cells could be expanded in vitro to decrease the number of donors needed per islet transplant, then encapsulated in humagel and transplanted to the patient. Another challenge to islet transplant and engineering is protecting the islets from the host's immune response. With a naturally derived membrane, the islets would be protected from the immune response of the host.

In other embodiments, humagel provides a safe method for delivering cells to target sites as well as providing the extracellular matrix to assist in cell proliferation and differentiation. The ability to induce secretion of growth factors, hormones and cytokines into the membrane through different stimulants allows us to design a membrane for the specific application. In this way, humagel could be used for treating spinal cord injuries. By encapsulating human mesenchymal stem cells (hMSCs) in the derived membrane, the cells could be safely delivered to the injury site. The hMSCs would be delivered to the site with the necessary growth factors as well as extracellular matrix for cell differentiation and proliferation, eventually the humagel would be degraded by the expanding cells and replaced with the naturally occurring ECM of the expanding cell population.

In certain other embodiments, humagel could also be used for bone tissue engineering applications. The humagel can be designed to promote bone tissue remodeling by increasing the amount of osteoprotegrin and osteopontin as well as increasing the collagen content in the gel for structural integrity. Humagel can then be used as a basement membrane for bone reconstruction. The humagel could be coupled with a polymer scaffold to promote bone growth in injured areas such as the face or legs or with increased collagen content as its own scaffold. The polymer scaffold would provide the structural integrity to fill the void of the injury while humagel was used to recruit and promote bone formation around the scaffolding.

In other embodiments, humagel may be used in wound healing applications. Wound healing is dependent on angiogenesis. As previously discussed, humagel is expected to promote angiogensis based on the mouse model and previous research. The advantage of the system of the present disclosure is that it is capable of producing a membrane from a human system allowing for the direct application of humagel to the wound. Applications of the present disclosure are predicated on the unique flexibility of the adipocytes to be used a bioreactor and manipulated to produce a designer extracellular matrix scaffold.

To facilitate a better understanding of the present invention, the following examples of certain aspects of some embodiments are given. In no way should the following examples be read to limit, or define, the entire scope of the invention.

### EXAMPLES

### Example 1

### Engineering a Human Adipocyte-Derived Extracellular Matrix Scaffold

Materials and Methods

Cell Culture: Simpson-Golabi-Behmel Syndrome preadipocytes were obtained from Dr. Martin Wabitsch. The SGBS cell line is grown in media, OF, containing DMEM nutrient mixture supplemented with 2% penicillin/streptomycin mixture, 10% fetal bovine serum, and 2.2 g sodium bicarbonate until confluency. The media is then changed to differentiation media, Day 0. The differentiation media consists of DMEM/F12 nutrient mix supplemented with 0.008 g biotin, 0.004 g pantothenate, 2% penicillin/streptomycin, 0.01 mg/mL human transferrin, 0.02 units/mL insulin, 100 nM cortisol, 0.2 nM T3, 25 nM dexamethasone, 50 μM 1-methyl-3-isobutylmethylxanthine (IBMX) and 2 μM rosiglitazone. The cells are cultured with the differentiation media for 4 days. On Day 4 the media is collected, labeled and stored at −20° C. The differentiation media is replaced with maintenance media consisting of DMEM/F12 mix supplemented with 0.008 g Biotin, 0.004 g pantothenate, 2% penicillin/streptomycin, 0.01 mg/mL human transferrin, 0.02 units/mL insulin, 100 nM cortisol, 0.2 nM T3 and 2 μM rosiglitazone. The media is then collected on days 8 and 12. The collected media is filtered using a 20 mL centrifuge filter for material greater than 100,000 MWCO or 100 kDa. A 100 kDa filter size was used to retain only the large ECM proteins secreted in to the exposed media. The retentate from this filtration is the concentrated material and is the gel used for different applications. The gel is then used as either a scaffold for cell culture or as a media supplement to promote cell adhesion. Images were acquired after 0, 4, 8 and 12 days of culture to verify differentiation efficiency with the accumulation of lipid droplets in the differentiated adipocytes.

Protein Content Determination: Protein content was determined using BCA assay (Pierce) with Bovine Serum Albumin (BSA) as standard. Briefly, 10 μL samples were added to 200 μL of reagent based on bicinchoninic acid, inducing a color change. The absorbance was then measured at 562 nm. The protein concentration in the sample was determined based on standard curves to yield total protein content of the samples.

Western Blot: Total material (10 μg/lane) was resolved by SDS-PAGE and transferred to PVDF membranes. A primary antibody against laminin (Santa Cruz) was used. Rabbit secondary antibodies were used at dilutions of 1:500.

Results

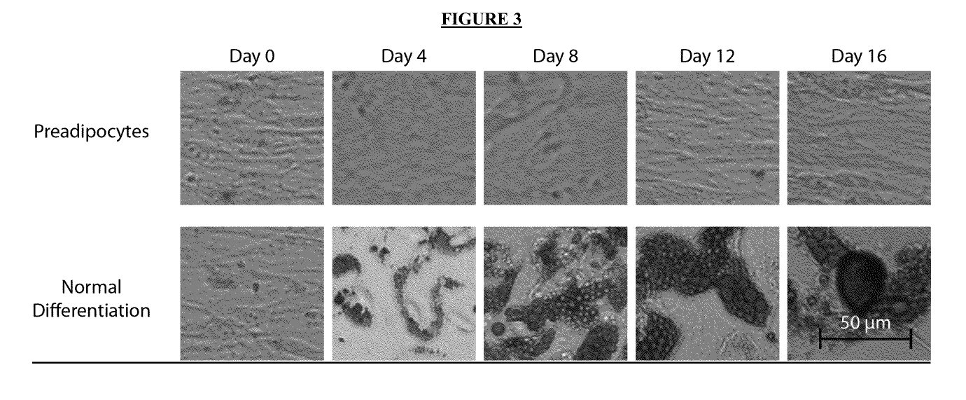

Adipocyte differentiation was confirmed through bright field imaging and Oil Red O staining of untreated preadipocytes (control) and treated preadipocytes (normal). Bright field imaging confirmed the morphology—a rounded shape with large lipid droplets throughout the cytoplasm- of the adipocytes, and revealed lipid droplet formation over the course of differentiation (FIG. 2). The treated preadipocytes stained positive for lipid droplets as seen in FIG. 3 while the untreated preadipocytes did not show staining for lipid droplets, further confirming adipocyte differentiation in the test group.

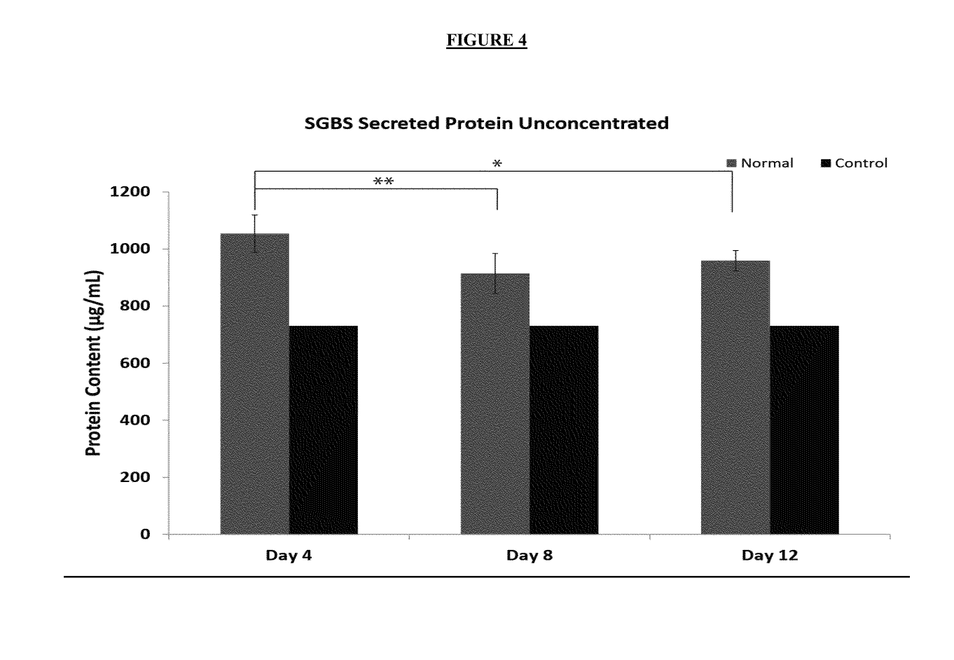

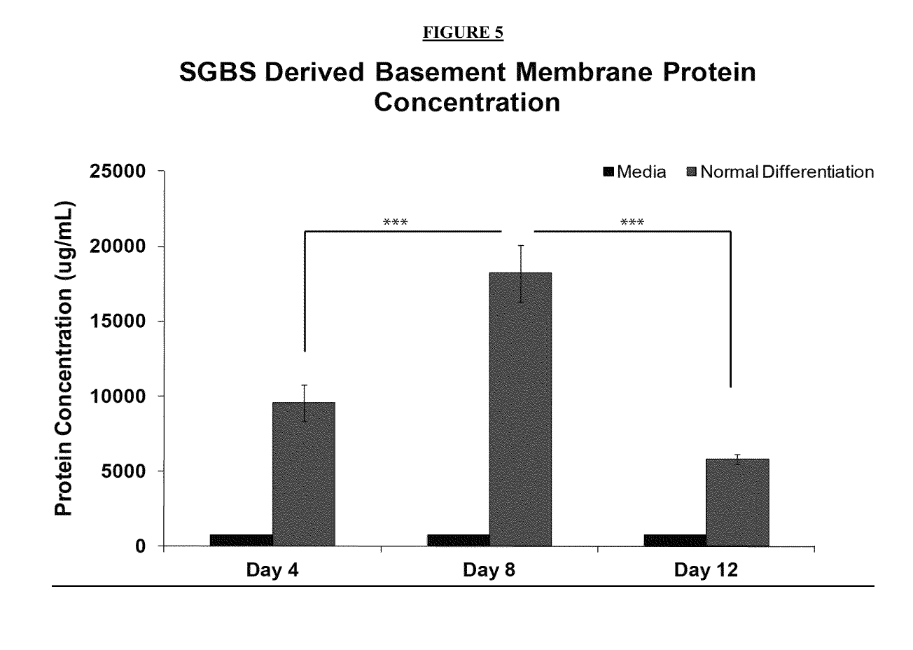

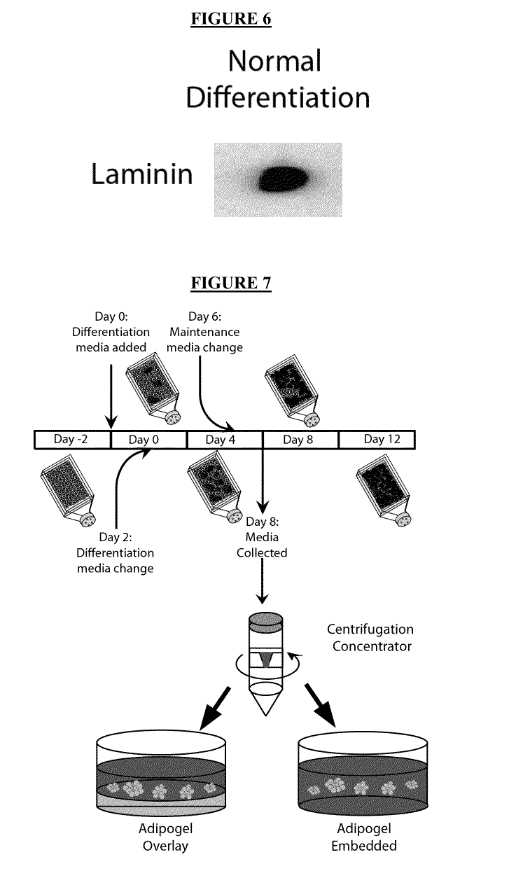

To determine the optimum collection day for secreted proteins, protein content was measured in the concentrated and unconcentrated media for days 4, 8 and 12. Significant material was obtained after collection and filtration. On average for days 4 and 12, 100 μL of material was obtained per 20 mL of media concentrated, while day 8 produced 200 μL of material per 20 mL of collected media. Protein measurements were made before and after filtration. Before filtration, all days had elevated levels of protein content when compared to the control media as seen in FIG. 4. Days 8 and 12 increased protein content to 913.76±70.5 μg/mL and 958.60±35.93 μg/mL respectively. Day 4 had the largest increase in protein content to 1053.38±65.33 μg/mL. After filtration, all days showed significant increase in protein content over the fresh unexposed media in FIG. 5. Days 4 and 12 increased protein content on average 8-fold over the normal media to 6,000 μg/mL. Day 8 showed the greatest increase in protein content to 22,500 μg/mL, an over 30-fold increase. To verify the presence of extracellular matrix proteins in the collected media, western blotting for laminin was performed. Western blotting revealed the presence of laminin, an extracellular matrix protein, in the filtered material as shown in FIG. 6.

The findings of this study indicate that the preadipocytes completely differentiated to mature adipocytes as seen by the Oil Red O staining and bright field imaging. Further, that during differentiation, the adipocytes secrete extracellular matrix proteins in varying amounts depending on the day of collection. Also, the secreted extracellular matrix proteins can be collected and filtered to yield a highly concentrated gel with greater than 20 times the protein found in normal media. Finally, the secreted matrix can be concentrated with potential use as a novel human-derived ECM scaffold, deemed humagel. Further work should focus on using different stimuli to increase and manipulate the composition of the secreted protein.

### Example 2

### Viability of Humagel as Tissue Engineering Scaffold

Materials and Methods

Islet Culture: Aliquots from healthy human islet isolations were cultured in a serum-free media (SFM) containing 1% ITS, 1% L-glutamine, 1% antibiotic antimyotic solution, and 16.8 μM/L zinc sulfate. Culture media was changed weekly during the culture period.

Islet Seeding: Type 1 collagen was prepared by extracting acid-soluble collagen from rat tail tendons. The control group was islets in suspension culture. For the single gel collagen and humagel overlay condition, 400 μL of a cold mixture, 9 parts 1.25 mg/mL rat tail collagen type 1 and 1 part 10× concentrated DMEM, was distributed in each well. For the double gel sandwich culture, 250 μL of a cold mixture, 9 parts 1.25 mg/mL rat tail collagen type 1 and 1 part 10× concentrated DMEM, was distributed in each well. For the embedded condition, 400 μL of a cold mixture, 9 parts 1.25 mg/mL rat tail collagen type 1 and 1 part 10× concentrated DMEM, was mixed directly with 100 islet equivalents and distributed in each well. To induce gelation, the coated plates were incubated for 60 minutes at 37° C. After gelation, the single gel wells were seeded with 100 islet equivalents (IEQ) each and 400 μL of CMRL media. The humagel overlay condition was seeded with 100 islet equivalents. After seeding, 50 μL of humagel was mixed with 400 μL of CMRL media by continuous pipeting and added to the humagel wells. For the embedded condition, 400 μL of CMRL media was added to each well. For the double gel sandwich culture, 400 μL of CMRL media was added to each well. After 48 hrs, the media was removed and 250 μL of collagen mixture was added to the double gel wells. To induce gelation, the coated plates were incubated for 60 minutes at 37° C. After gelation, 400 μL of media was added to the well. The media was collected and changed on Days 7 and 14.

Glucose Stimulated Insulin Release Test: The in vitro function of human islets was determined by measuring secreted human insulin using an ELISA kit (ALPCO Diagnostics, Windham, N.H.). The islets were incubated at 37° C. for 1 hr with MEM media containing low (0.6 mg/ml, basal) and high (3 mg/ml, stimulated) glucose concentrations. After incubation, supernatants were collected and analyzed for insulin release. Insulin secretion was expressed as μU/mL and the ratio of insulin level at 3 mg/mL glucose to the insulin level at 0.6 mg/mL glucose was used to calculate the stimulation index (SI).

Islet Morphology: Islet morphology was studied by light microscope photography. Pictures were taken using a camera at 20× magnification on days 1, 7 and 14.

Results

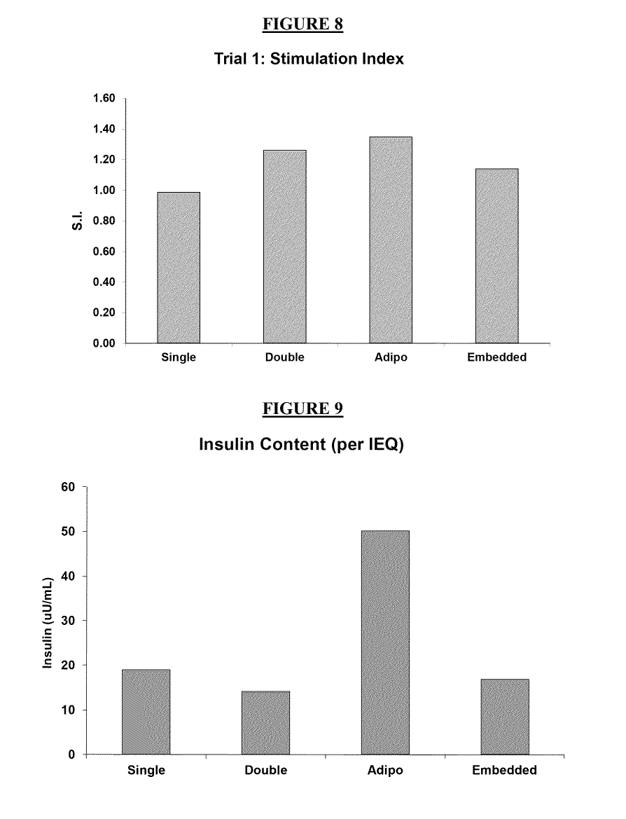

To determine the effect of humagel on islet function maintenance in culture, we measured the stimulation index and insulin content as well as observing the morphology. The stimulation index (SI) is a measure of islet function calculated by dividing the insulin released at high glucose concentrations by the insulin released at low glucose concentrations. An SI index greater than one signifies that the islets have retained their function and still respond to changes in glucose concentration, while an SI less than one is indicative of nonresponsive islets that have lost their function. The stimulation index for islets cultured in humagel overlay was measured on Day 7. The first trial was only 2 wells per condition as a proof of concept study. The Day 7 measurement was 24 days post isolation. The control group was islets in suspension culture with CMRL media. The SI was measured for islets cultured on a single collagen layer, double collagen layer, embedded in collagen, and single gel collagen with humagel overlay as shown in FIG. 8. The SI for humagel was 1.35, double gel 1.26, embedded 1.14, control 1.13, and the single gel was 0.99. FIG. 9 shows the insulin content per islet equivalent (IEQ) measured in microunits per milliliter (μU/mL). Insulin content for the control group and humagel overlay conditions were significantly higher than the other conditions at 58.6 μU/mL and 50.2 μU/mL respectively. The single collagen gel was 18.9 μU/mL, with the embedded islet gel insulin content at 16.8 μU/mL and the double gel insulin content at 14.1 μU/mL.

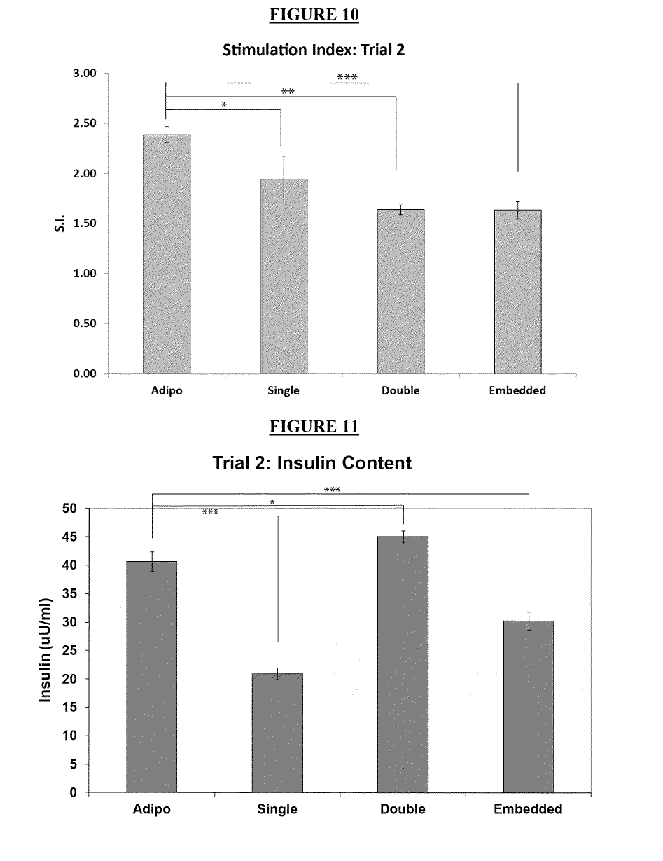

The stimulation index (SI) for islets cultured in humagel was measured on Day 7. The second trial used 3 wells per condition for stimulation index at 100 IEQ as well as 3 wells for insulin content at 50 IEQ. The Day 7 measurement was 12 days post isolation. The control group was islets in suspension culture. The SI was measured for islets cultured on a single collagen layer, double collagen layer, embedded in collagen, and single gel collagen with humagel overlay as shown in FIG. 10. The SI for humagel was 2.39, single gel was 1.94, double gel 1.64, and embedded 1.63. Fresh isolated islets had a stimulation index of 7.95. FIG. 11 shows the insulin content per islet equivalent (IEQ) measured in microunits per milliliter (μU/mL). Insulin content for the humagel overlay was second highest in the second trial, at 40.64±5.9 μU/mL, while the highest insulin content was the double gel at 45.0±3.6 μU/mL. The embedded condition and single collagen layer had an insulin content of 30.24±5.5 μU/mL and 20.9±3.5 μU/mL, respectively.

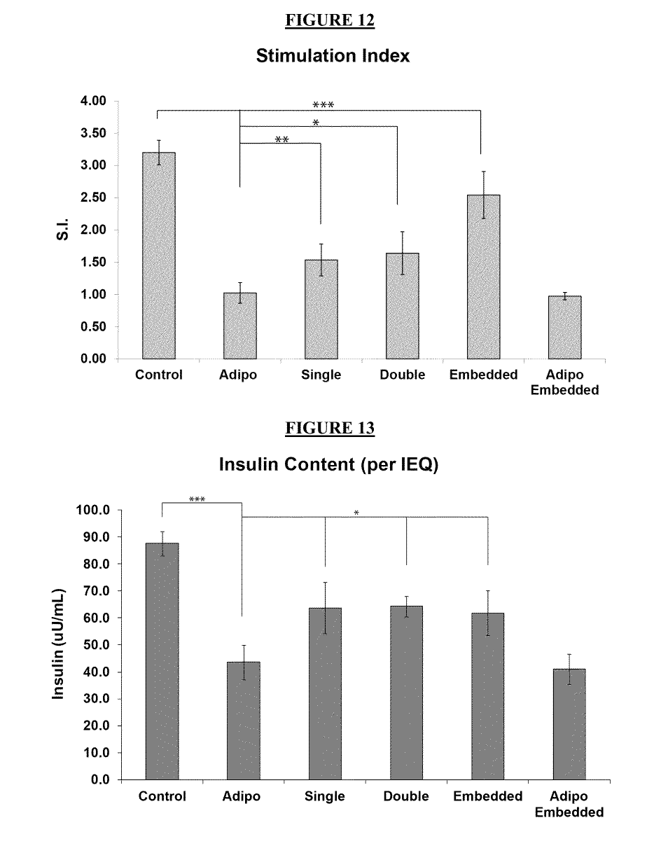

The stimulation index (SI) for islets cultured in humagel was measured on Day 7. The third trial was 3 wells per condition for stimulation index at 100 IEQ as well as 3 wells for insulin content at 50 IEQ. The Day 7 measurement was 13 days post isolation. The control group was islets in suspension culture. The SI was measured for islets cultured on a single collagen layer, double collagen layer, embedded in collagen, and single gel collagen with humagel overlay as shown in FIG. 12. A new condition was introduced for the third trial; islets were embedded in a combination of humagel and collagen at a ratio of 2:3. The SI for control group was 3.20±0.19, single gel was 1.53±0.25, double gel 1.64±0.33, and embedded 2.54±0.36. The humagel performed the worst in the trial with an SI of 1.02±0.16 for the humagel overlay condition and 0.97±0.06 for the humagel-collagen mixture. FIG. 13 shows the insulin content per islet equivalent (IEQ) measured in microunits per milliliter (μU/mL). Insulin content for the control group was 87.6±4.5 μU/mL. The single gel, double gel and embedded conditions all had similar insulin content at 63.7±9.5 μU/mL, 64.3±3.7 μU/mL, and 61.9±8.3 μU/mL. The humagel overlay and humagel-collagen mixture were significantly lower at 43.6±6.3 μU/mL and 41.1±5.5 μU/mL, respectively.

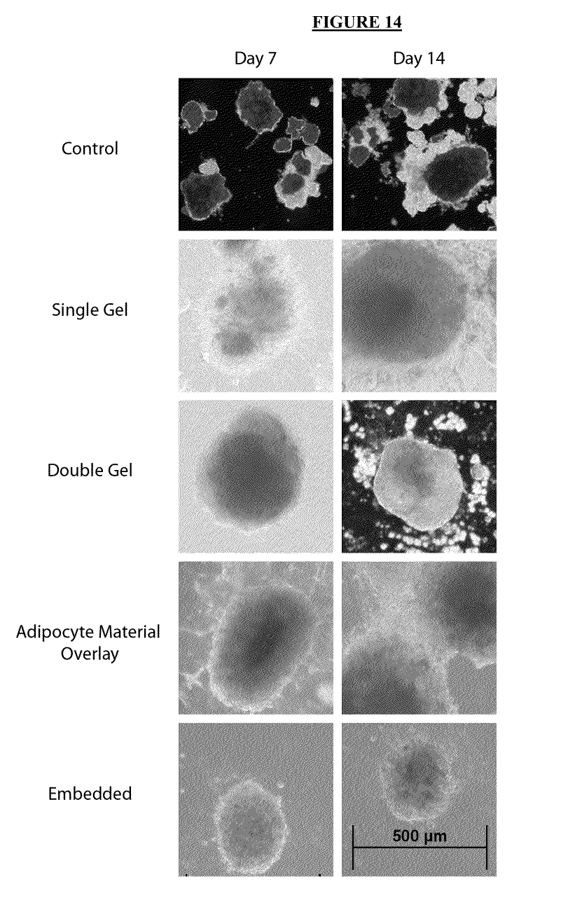

Since functionality of islets is closely tied to islet morphology, we studied the morphology of islets using light microscope photography at 20× magnification, FIG. 14. The control group is the suspension culture and shows islets with their solid edges intact on day 7 and 14. The single collagen gel islets showed only partial morphology breakdown on day 7 and significant morphology changes on day 14 as seen by the lines extruding from original islet borders. The double collagen gel and embedded condition both maintained islet morphology with slight changes seen on day 14. The humagel overlay shows significant changes in morphology on day 7 and day 14. On day 7 there is a noticeable spidering effect coming from the islets, and on day 14 the effect has nearly interconnected islets. Some islets in humagel overlay showed an emptying of the islets with significant cell numbers extending from the empty shell, as seen in FIG. 14.

When islets were cultured in the presence of the newly formed humagel, islet functionality increased. However, when the digestion process was modified in an attempt to increase the effect of humagel, there was a decrease in humagel effectiveness that may be attributed to the digestion modification or a poor differentiation of preadipocytes. Further, islets cultured in the presence of humagel experienced a more rapid break down in morphology showing increased cell migration out of the islet. However, the cells around the islets may be newly formed insulin-producing β-cells with significant implications for islet tissue engineering and transplantation. Further work should focus on confirming that the cells appearing around the islet in vitro are β-cells.

### Example 3

### Humagel for Soft Tissue Reconstruction

Materials and Methods

Adipogenic Induction Potential: To test the adipogenic induction potential of the humagel, SGBS cells were seeded in 24-well tissue culture plates. The cells were allowed to grow to two days post-confluency. At this time, four groups were created. The first group was differentiated using the previously described protocol. To account for self-induced differentiation, another group was cultured in OF media without supplements. A third group was cultured using maintenance media supplemented with 2 μM Rosiglitazone. The final group consisted of cells differentiated using maintenance media supplemented with 50 μL of humagel. The cells were then stained for lipid accumulation using Oil Red O. For Oil Red O (Sigma-Aldrich) staining, the adherent cells were fixed in 10% formalin, washed and stained with a 0.21% (w/v) Oil Red O solution (60% isopropanol and 40% water). Oil Red O staining was then analyzed using bright field imaging. Images were acquired using an EVOS microscope at 20× magnification.

Results

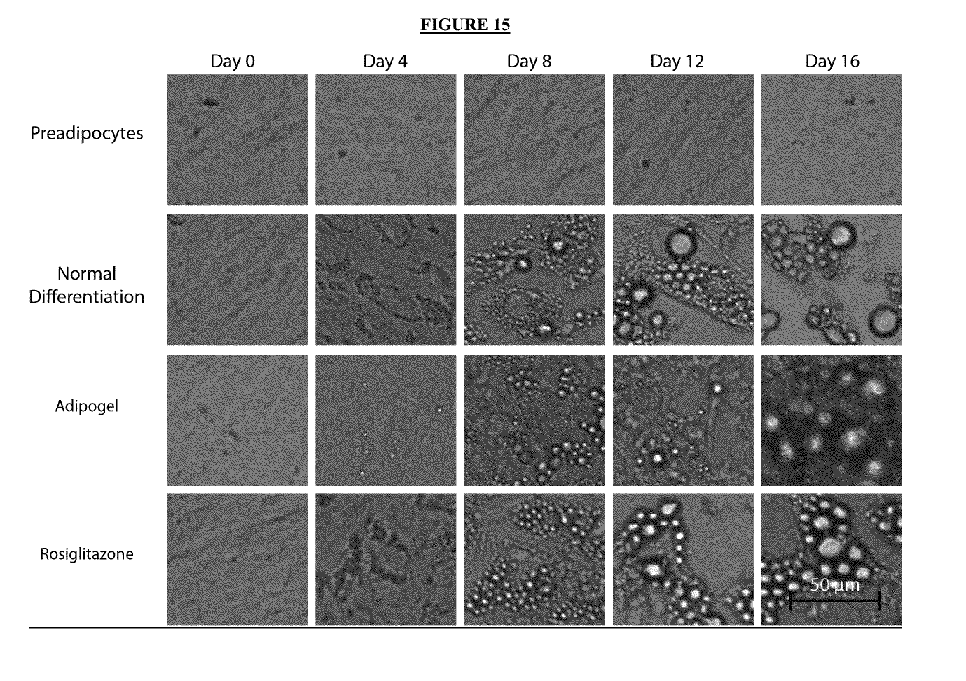

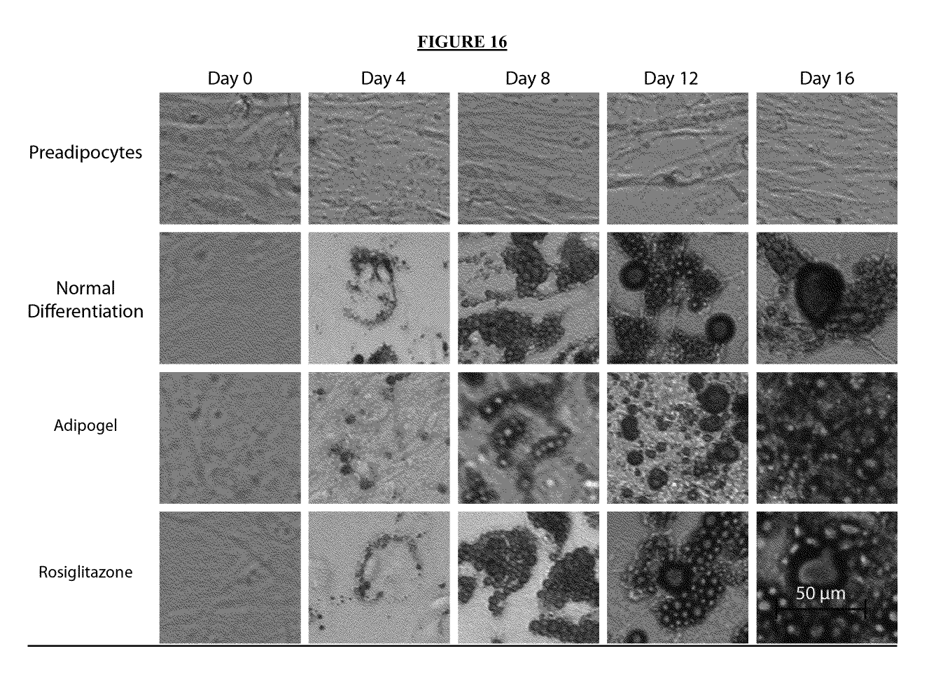

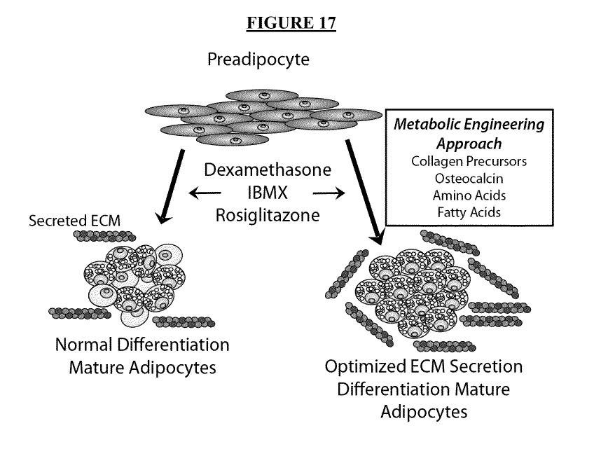

To determine the adipogenic differentiation potential of humagel, we used humagel as a media supplement for confluent SGBS preadipocytes. Bright field imaging and Oil Red O staining were used to determine the adipogenesis induction potential of the filtered material. The bright field images shown in FIG. 15 displayed no droplet formation in the untreated preadipocytes. In the normal treated adipocytes, lipid droplets could be observed starting on day 4. For the humagel overlay, lipid droplets began to form on day 4 but did not mature in the same way as the treated adipocytes. The untreated preadipocytes served as a control and did not stain for lipid droplets. The treated (normal) preadipocytes stained positive for lipid droplets using Oil Red O staining, starting on day 4, and continued to develop larger lipid droplets over the course of differentiation. Staining of the humagel overlay treated preadipocytes confirmed the presence of lipid droplets beginning on day 4 and continuing through the differentiation. However, the humagel treated preadipocytes showed an increased number of smaller lipid droplets on day 16 than the normal differentiation preadipocytes, as seen in FIG. 16.

The humagel was capable of inducing differentiation in SGBS preadipocytes. With the potential to induce differentiation the humagel can be used in a variety of soft tissue repair situations such as soft tissue defects or breast reconstruction after mastectomy. Further, the humagel-treated adipocytes yielded smaller lipid droplet sizes that suggest a higher sensitivity to insulin than adipocytes treated with rosiglitazone or the normal differentiation solution. Future work on this subject should focus on using humagel as a scaffold for growing and differentiating preadipocytes, as well as determining if humagel is effective for differentiating human mesenchymal stem cells (hMSCs) into mature adipocytes.

### Example 4

### Cells as a Bioreactor for Tissue Engineering Scaffolds

Materials and Methods

Cell Culture: Cells are expanded as previously described in chapter 2 and seeded in 24-well tissue culture plates. Two days after the cells have reached confluency, the differentiation media is added. The cells are cultured with the differentiation media for 4 days. On Day 4 the media is collected, labeled and stored at −20° C. The differentiation media is replaced with maintenance media consisting of DMEM/F12 mix supplemented with 0.008 g biotin, 0.004 g pantothenate, 2% penicillin/streptomycin, 0.01 mg/mL human transferrin, 0.02 units/mL insulin, 100 nM cortisol, 0.2 nM T3 and 2 μM rosiglitazone for the control group. For the first condition the maintenance media is supplemented with 2 μM rosiglitazone. The Amino Acid 1(AA1) group consisted of maintenance media supplemented with 3 mg/mL proline. 5 nM osteocalcin was added to the maintenance media for the group labeled Ost. The second Amino acid group, AA2, consisted of 3 mg/mL hydroxyproline added to the maintenance media. The fatty acid groups consisted of oleic acid, palmitic acid, and linoleic acid added to maintenance mediagroups individually at a concentration of 50 μM. The media is then collected on days 6, 8, and 10. The collected media is labeled and stored at −20° C. for further analysis.

Protein Content Determination: Protein content was determined using BCA assay (Pierce) with Bovine Serum Albumin (BSA) as standard. Briefly, 10 μL samples were added to 200 μL of reagent based on bicinchoninic acid, inducing a color change. The absorbance was then measured at 562 nm. The protein concentration in the sample was determined based on standard curves to yield total protein content of the samples.

Oil Red Staining: For Oil Red O (Sigma-Aldrich) staining, adherent cells were fixed in 10% formalin, washed and stained with a 0.21% (w/v) Oil Red O solution (60% isopropanol and 40% water). Oil Red O staining was then analyzed using bright field imaging. Images were acquired using an EVOS microscope at 20× magnification.

Results

To assess adipocyte differentiation, bright field images were taken of Oil Red stained and unstained cells after treatment with adipogenic factors. Bright field images taken on day 10 of the treated preadipocytes revealed the formation of large lipid droplets in all treated preadipocyte groups. The control group did not show any lipid formation. The linoleic acid group showed cell detachment. Oil Red staining confirmed the existence of lipid droplets in all test groups, with the largest percentage of differentiation occurring in the oleic and palmitic acid groups, and the least in the linoleic acid group. The control group showed no staining, indicating that all differentiation was a result of treatment and there was no self-induced differentiation. Finally, while the stressed condition was a promising idea similar to flow stress, the adipocytes failed to reattach after treatment and died.

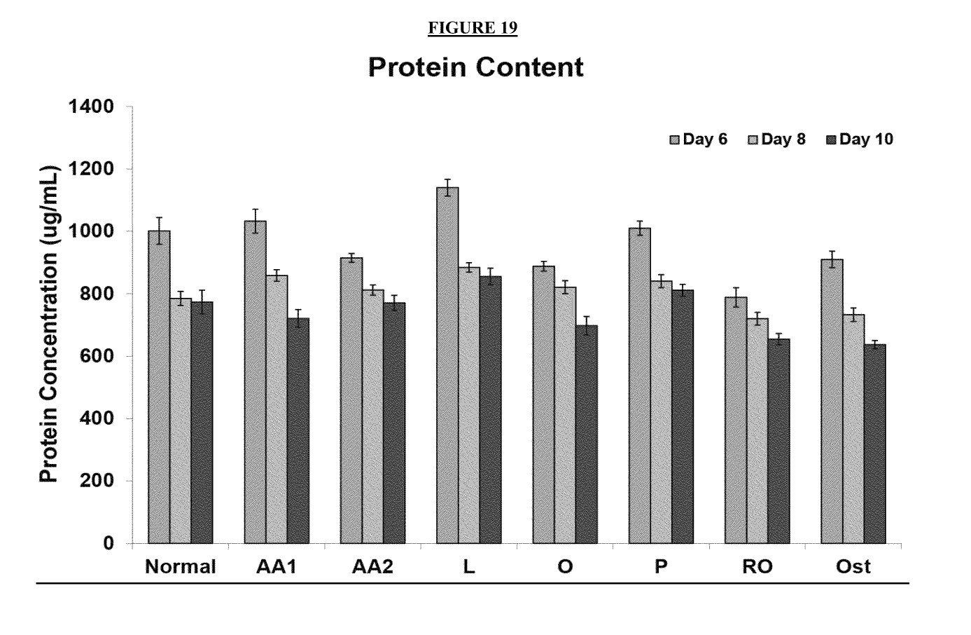

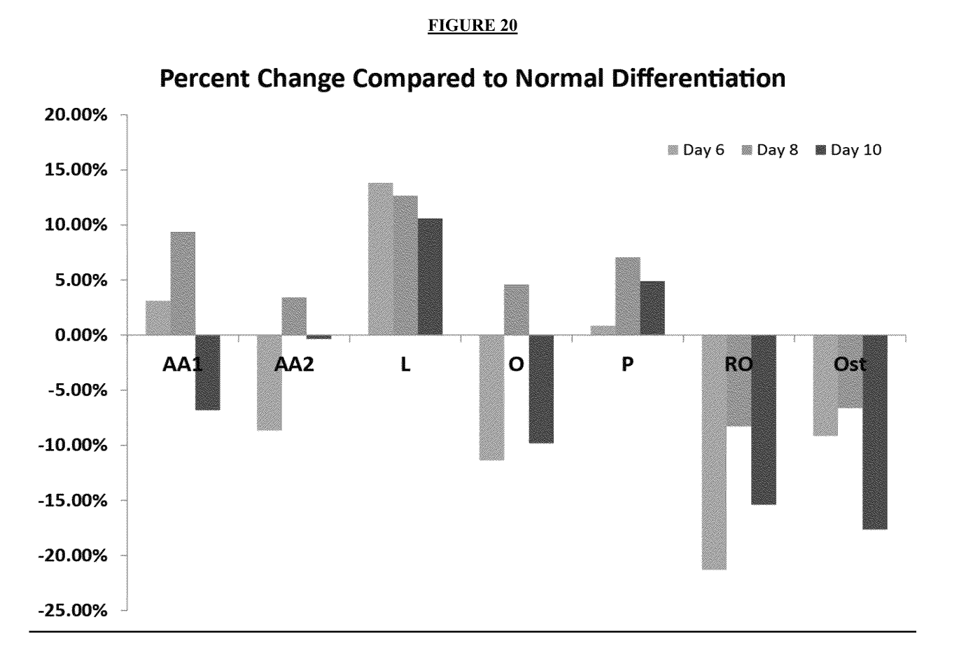

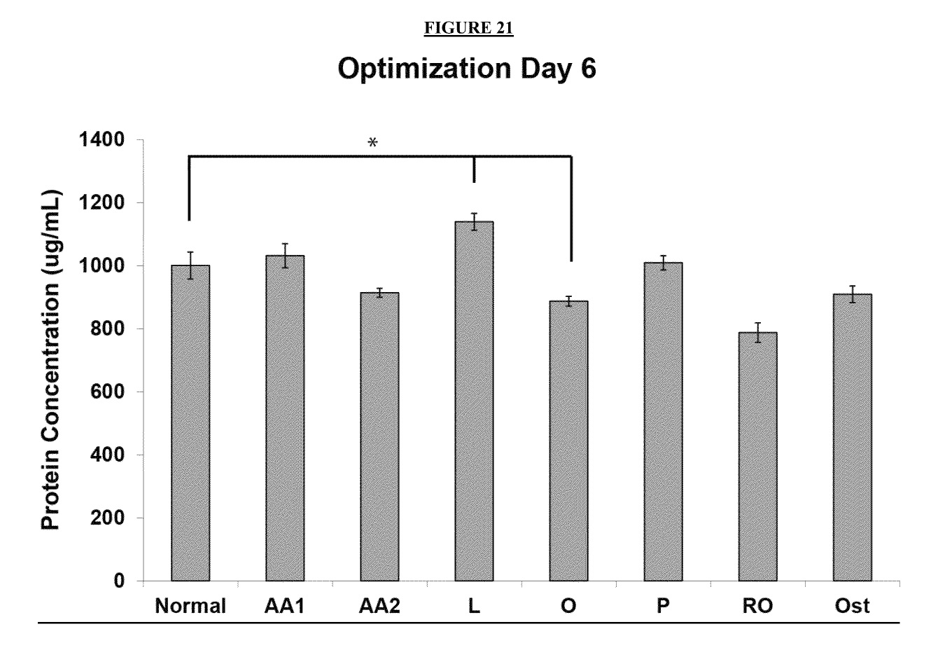

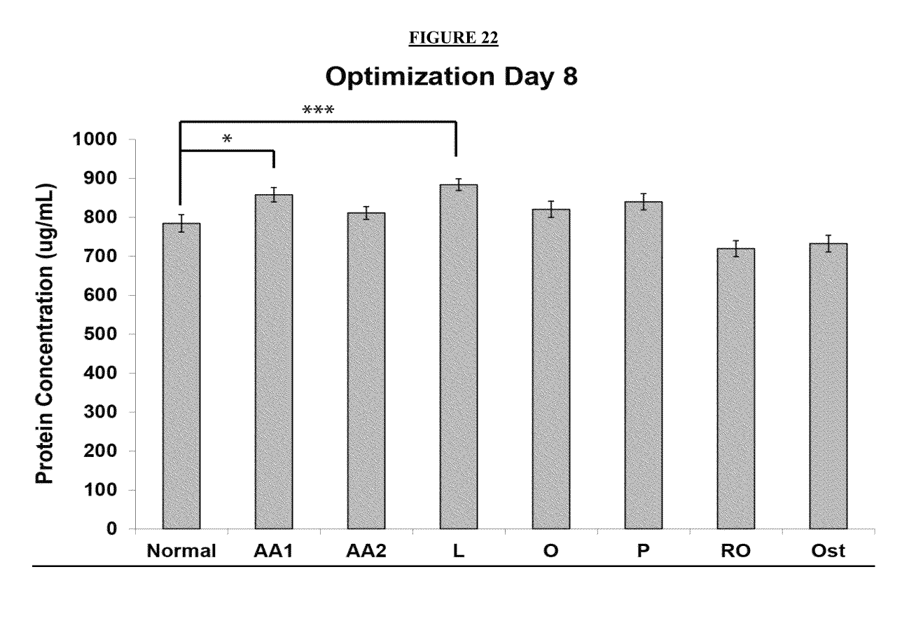

To determine if there was a significant change in protein production in adipocytes treated with different media supplements during differentiation, we measured protein content on Days 6, 8 and 10. Days 2 and 4 were not measured because all conditions were treated with the same differentiation-inducing media during these days. Protein content measurements revealed a negative slope in protein content over the course of differentiation, with the highest point generally on day 6 and decreasing over the remaining days as seen in FIG. 19 with error values expressed as SEM. There is an increase in protein production on Day 6 between the linoleic acid supplemented differentiation, 1139.8 μg/mL, and the normal differentiation, 1001.35 μg/mL. There was also a significant decrease in protein secretion in the oleic acid supplemented media to 887.71 μg/mL. On Day 8, the proline supplemented media, AA1, showed a marked increase in protein content over the normal differentiation with a value of 858.4 μUg/mL as opposed to 784.8 μg/mL for the normal differentiation. Also, linoleic acid continued to show an increase in protein secretion on Day 8 with a value of 884.1 μg/mL. Further evaluation of protein secretion in terms of percent change compared to normal shows that only linoleic acid significantly increased production content in the media over the test period, FIG. 20. FIGS. 21 and 22 show a detailed comparison amongst the groups on Day 6 and 8 respectively with statistical analysis.

The findings of this study demonstrate the ability to modulate the amount of protein secreted from the adipocytes during differentiation based on nutritional and hormonal supplements. From this testing, the most effective stimulants for increasing protein production were proline, oleic acid, and linoleic acid. Further through this method, we have shown that adipocytes, and possibly other cells with ECM protein synthesis pathways, may be used as bioreactors for engineering designer tissue-scaffolds based on specific application constraints. Future work in this topic should focus on characterizing the proteins secreted during treatment and the use of different cell types to generate a specific ECM microenvironment.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of or “consist of the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

### REFERENCES

1. Langer, R. and J. P. Vacanti, Tissue engineering. Science, 1993. 260(5110): p. 920-6.
2. Vacanti, J. P., et al., Selective cell transplantation using bioabsorbable artificial polymers as matrices. J Pediatr Surg, 1988. 23(1 Pt 2): p. 3-9.
3. Athanasiou, K. A., et al., Orthopaedic applications for PLA-PGA biodegradable polymers. Arthroscopy, 1998. 14(7): p. 726-37.
4. Uygun, B. E., et al., Organ reengineering through development of a transplantable recellularized liver graft using decellularized liver matrix. Nat Med, 2010. 16(7): p. 814-20.
5. Grauss, R. W., et al., Decellularization of rat aortic valve allografts reduces leaflet destruction and extracellular matrix remodeling. J Thorac Cardiovasc Surg, 2003. 126(6): p. 2003-10.
6. De Carlo, E., et al., Pancreatic acellular matrix supports islet survival and function in a synthetic tubular device: in vitro and in vivo studies. Int J Mol Med, 2010. 25(2): p. 195-202.
7. Baptista, P. M., et al., The use of whole organ decellularization for the generation of a vascularized liver organoid. Hepatology, 2011. 53(2): p. 604-17.
8. Wolchok, J. C. and P. A. Tresco, The isolation of cell derived extracellular matrix constructs using sacrificial open-cell foams. Biomaterials, 2010. 31(36): p. 9595-603.
9. Datta, N., et al., Effect of bone extracellular matrix synthesized in vitro on the osteoblastic differentiation of marrow stromal cells. Biomaterials, 2005. 26(9): p. 971-7.
10. Buechter, D. D., et al., Co-translational incorporation of trans-4-hydroxyproline into recombinant proteins in bacteria. J Biol Chem, 2003. 278(1): p. 645-50.
11. Keizer-Gunnink, I., et al., Accumulation of properly folded human type III procollagen molecules in specific intracellular membranous compartments in the yeast Pichia pastoris. Matrix Biol, 2000. 19(1): p. 29-36.
12. Fukuda, K., et al., Formation of recombinant triple-helical [alpha 1(IV)]2 alpha 2 (IV) collagen molecules in CHO cells. Biochem Biophys Res Commun, 1997. 231(1): p. 178-82.
13. Mazzorana, M., et al., Mechanisms of collagen trimer formation. Construction and expression of a recombinant minigene in HeLa cells reveals a direct effect of prolyl hydroxylation on chain assembly of type XII collagen. J Biol Chem, 1993. 268(5): p. 3029-32.
14. Ruggiero, F. and M. Koch, Making recombinant extracellular matrix proteins. Methods, 2008. 45(1): p. 75-85.
15. Uitto, J. and J. R. Lichtenstein, Defects in the biochemistry of collagen in diseases of connective tissue. J Invest Dermatol, 1976. 66(02): p. 59-79.
16. Lu, H., et al., Cultured cell-derived extracellular matrix scaffolds for tissue engineering. Biomaterials, 2011. 32(36): p. 9658-66.
17. Sharma, N. S., D. Nagrath, and M. L. Yarmush, Adipocyte-derived basement membrane extract with biological activity: applications in hepatocyte functional augmentation in vitro. FASEB J, 2010. 24(7): p. 2364-74.
18. Okano, T., et al., A novel recovery system for cultured cells using plasma-treated polystyrene dishes grafted with poly(N-isopropylacrylamide). J Biomed Mater Res, 1993. 27(10): p. 1243-51.
19. Kushida, A., et al., Decrease in culture temperature releases monolayer endothelial cell sheets together with deposited fibronectin matrix from temperature-responsive culture surfaces. J Biomed Mater Res, 1999. 45(4): p. 355-62.
20. Shimizu, H., et al., Bioengineering of a functional sheet of islet cells for the treatment of diabetes mellitus. Biomaterials, 2009. 30(30): p. 5943-9.
21. El Ghalbzouri, A., et al., Replacement of animal-derived collagen matrix by human fibroblast-derived dermal matrix for human skin equivalent products. Biomaterials, 2009. 30(1): p. 71-8.
22. Datta, N., et al., In vitro generated extracellular matrix and fluid shear stress synergistically enhance 3D osteoblastic differentiation. Proc Natl Acad Sci USA, 2006. 103(8): p. 2488-93.
23. Decaris, M. L., et al., Transferable cell-secreted extracellular matrices enhance osteogenic differentiation. Acta Biomater, 2012. 8(2): p. 744-52.
24. Wabitsch, M., et al., Characterization of a human preadipocyte cell strain with high capacity for adipose differentiation. Int J Obes Relat Metab Disord, 2001. 25(1): p. 8-15.
25. Rosenow, A., et al., Identification of novel human adipocyte secreted proteins by using SGBS cells. J Proteome Res, 2010. 9(10): p. 5389-401.
26. Wild, S., et al., Global prevalence of diabetes: estimates for the year 2000 and projections for 2030. Diabetes Care, 2004. 27(5): p. 1047-53.
27. Blomeier, H., et al., Polymer scaffolds as synthetic microenvironments for extrahepatic islet transplantation. Transplantation, 2006. 82(4): p. 452-9.
28. Ryan, E. A., et al., Five-year follow-up after clinical islet transplantation. Diabetes, 2005. 54(7): p. 2060-9.
29. Giraldo, J. A., J. D. Weaver, and C. L. Stabler, Tissue engineering approaches to enhancing clinical islet transplantation through tissue engineering strategies. J Diabetes Sci Technol, 2010. 4(5): p. 1238-47.
30. Beck, J., et al., Islet encapsulation: strategies to enhance islet cell functions. Tissue Eng, 2007. 13(3): p. 589-99.
31. Lucas-Clerc, C., et al., Long-term culture of human pancreatic islets in an extracellular matrix: morphological and metabolic effects. Mol Cell Endocrinol, 1993. 94(1): p. 9-20.
32. Daoud, J., et al., The effect of extracellular matrix components on the preservation of human islet function in vitro. Biomaterials, 2010. 31(7): p. 1676-82.
33. Daoud, J. T., et al., Long-term in vitro human pancreatic islet culture using three-dimensional microfabricated scaffolds. Biomaterials, 2011. 32(6): p. 1536-42.
34. Lai, Y., et al., Vascular endothelial growth factor increases functional beta-cell mass by improvement of angiogenesis of isolated human and murine pancreatic islets. Transplantation, 2005. 79(11): p. 1530-6.
35. Cheng, J. Y., et al., Matrix components and scaffolds for sustained islet function. Tissue Eng Part B Rev, 2011. 17(4): p. 235-47.
36. Kaido, T., et al., Impact of defined matrix interactions on insulin production by cultured human beta-cells: effect on insulin content, secretion, and gene transcription. Diabetes, 2006. 55(10): p. 2723-9.
37. Weinberg, N., et al., Lineage tracing evidence for in vitro dedifferentiation but rare proliferation of mouse pancreatic beta-cells. Diabetes, 2007. 56(5): p. 1299-304.
38. Surgeons, A.S.o.P. Reconstructive Surgery Procedures Statistics. 2008.
39. Patrick, C. W., Jr., Tissue engineering strategies for adipose tissue repair. Anat Rec, 2001. 263(4): p. 361-6.
40. Rohrich, R. J., E. S. Sorokin, and S. A. Brown, In search of improved fat transfer viability: a quantitative analysis of the role of centrifugation and harvest site. Plast Reconstr Surg, 2004. 113(1): p. 391-5; discussion 396-7.
41. Billings, E., Jr. and J. W. May, Jr., Historical review and present status of free fat graft autotransplantation in plastic and reconstructive surgery. Plast Reconstr Surg, 1989. 83(2): p. 368-81.
42. Patrick, C. W., Jr., Adipose tissue engineering: the future of breast and soft tissue reconstruction following tumor resection. Semin Surg Oncol, 2000. 19(3): p. 302-11.
43. Katz, A. J., et al., Emerging approaches to the tissue engineering of fat. Clin Plast Surg, 1999. 26(4): p. 587-603, viii.
44. Beahm, E. K., R. L. Walton, and C. W. Patrick, Jr., Progress in adipose tissue construct development. Clin Plast Surg, 2003. 30(4): p. 547-58, viii.
45. Gentleman, E., et al., Collagen composite biomaterials resist contraction while allowing development of adipocytic soft tissue in vitro. Tissue Eng, 2006. 12(6): p. 1639-49.
46. Cho, S. W., et al., Engineering of volume-stable adipose tissues. Biomaterials, 2005. 26(17): p. 3577-85.
47. Hemmrich, K., et al., Implantation of preadipocyte-loaded hyaluronic acid-based scaffolds into nude mice to evaluate potential for soft tissue engineering. Biomaterials, 2005. 26(34): p. 7025-37.
48. De Ugarte, D. A., et al., Future of fat as raw material for tissue regeneration Ann Plast Surg, 2003. 50(2): p. 215-9.
49. Gomillion, C. T. and K. J. Burg, Stem cells and adipose tissue engineering. Biomaterials, 2006. 27(36): p. 6052-63.
50. Lundgren, M., et al., Fat cell enlargement is an independent marker of insulin resistance and ‘hyperleptinaemia’. Diabetologia, 2007. 50(3): p. 625-33.
51. Vallee, M., J. F. Cote, and J. Fradette, Adipose-tissue engineering: taking advantage of the properties of human adipose-derived stem/stromal cells. Pathol Biol (Paris), 2009. 57(4): p. 309-17.
52. Vermette, M., et al., Production of a new tissue-engineered adipose substitute from human adipose-derived stromal cells. Biomaterials, 2007. 28(18): p. 2850-60.
53. Flynn, L. E., The use of decellularized adipose tissue to provide an inductive microenvironment for the adipogenic differentiation of human adipose-derived stem cells. Biomaterials, 2010. 31(17): p. 4715-24.
54. Bancroft, G. N., et al., Fluid flow increases mineralized matrix deposition in 3D perfusion culture of marrow stromal osteoblasts in a dose-dependent manner. Proc Natl Acad Sci USA, 2002. 99(20): p. 12600-5.
55. Swartz, M. A., et al., Mechanical stress is communicated between different cell types to elicit matrix remodeling. Proc Natl Acad Sci USA, 2001. 98(11): p. 6180-5.
56. Sarasa-Renedo, A. and M. Chiquet, Mechanical signals regulating extracellular matrix gene expression in fibroblasts. Scand J Med Sci Sports, 2005. 15(4): p. 223-30.
57. Chiquet, M., Regulation of extracellular matrix gene expression by mechanical stress. Matrix Biol, 1999. 18(5): p. 417-26.
58. Wei, Z., et al., Analysis and interpretation of stress fiber organization in cells subject to cyclic stretch. J Biomech Eng, 2008. 130(3): p. 031009.
59. Riboh, J., et al., Optimization of flexor tendon tissue engineering with a cyclic strain bioreactor. J Hand Surg Am, 2008. 33(8): p. 1388-96.
60. Ferron, M., et al., Insulin signaling in osteoblasts integrates bone remodeling and energy metabolism. Cell, 2010. 142(2): p. 296-308.
61. Ferron, M., et al., Osteocalcin differentially regulates beta cell and adipocyte gene expression and affects the development of metabolic diseases in wild-type mice. Proc Natl Acad Sci USA, 2008. 105(13): p. 5266-70.
62. Nimni, M. E., Collagen: structure, function, and metabolism in normal and fibrotic tissues. Semin Arthritis Rheum, 1983. 13(1): p. 1-86.
63. Nimni, M., Methods and compositions for enhancing collagen and proteoglycan synthesis in the skin. 2009: United States.
64. Han, B. and M. E. Nimni, Transdermal delivery of amino acids and antioxidants enhance collagen synthesis: in vivo and in vitro studies. Connect Tissue Res, 2005. 46(4-5): p. 251-7.
65. Han, B., et al., Proanthocyanidin: a natural crosslinking reagent for stabilizing collagen matrices. J Biomed Mater Res A, 2003. 65(1): p. 118-24.
66. Dioguardi, F. S., Compositions based on amino acids for preventing and treating precursor deficiencies in the sythesis of collagen. 1993: United States.
67. Keeping, H. S., Methods for the production of biologically active agents contained in an extracellular matrix. 2001: United States.
68. Shibayama, H., et al., Effect of a novel ascorbic derivative, disodium isostearyl 2-O-L-ascorbyl phosphate on human dermal fibroblasts: increased collagen synthesis and inhibition of MMP-1. Biol Pharm Bull, 2008. 31(4): p. 563-8.
69. Senturk, N., et al., The role of ascorbic acid on collagen structure and levels of serum interleukin-6 and tumour necrosis factor-alpha in experimental lathyrism. Clin Exp Dermatol, 2004. 29(2): p. 168-75.
70. Satory, D. L. and S. B. Smith, Conjugated linoleic acid inhibits proliferation but stimulates lipid filling of murine 3T3-L1 preadipocytes. J Nutr, 1999. 129(1): p. 92-7.
71. Xie, W., et al., Oleate-induced formation of fat cells with impaired insulin sensitivity. Lipids, 2006. 41(3): p. 267-71.
72. Madsen, L., R. K. Petersen, and K. Kristiansen, Regulation of adipocyte differentiation and function by polyunsaturated fatty acids. Biochim Biophys Acta, 2005. 1740(2): p. 266-86.
73. Madsen, L., et al., Adipocyte differentiation of 3T3-L1 preadipocytes is dependent on lipoxygenase activity during the initial stages of the differentiation process. Biochem J, 2003. 375(Pt 3): p. 539-49.
74. Rustad, K. C., et al., Strategies for organ level tissue engineering. Organogenesis, 2010. 6(3): p. 151-7.
75. Listenberger, L. L., et al., Triglyceride accumulation protects against fatty acid-induced lipotoxicity. Proc Natl Acad Sci USA, 2003. 100(6): p. 3077-82.
76. Sabin, M. A., et al., Depot-specific effects of fatty acids on lipid accumulation in children's adipocytes. Biochem Biophys Res Commun, 2007. 361(2): p. 356-61.
77. Murakami, H., Amino Acid composition promoting collagen synthesis, U.S.P. Office, Editor. 2010, Ajinomoto Co., Inc., Tokyo: United States of America.
78. Chavey, C., et al., Matrix metalloproteinases are differentially expressed in adipose tissue during obesity and modulate adipocyte differentiation. J Biol Chem, 2003. 278(14): p. 11888-96.
79. Jones, J. A., et al., Matrix metalloproteinases and their inhibitors in the foreign body reaction on biomaterials. J Biomed Mater Res A, 2008. 84(1): p. 158-66.
80. Anderson, J. M., A. Rodriguez, and D. T. Chang, Foreign body reaction to biomaterials. Semin Immunol, 2008. 20(2): p. 86-100.
81. Yang, J., et al., Cell sheet engineering: recreating tissues without biodegradable scaffolds. Biomaterials, 2005. 26(33): p. 6415-22.
82. Williams, D. F., On the mechanisms of biocompatibility. Biomaterials, 2008. 29(20): p. 2941-53.
83. Nichols, J. E. and J. Cortiella, Engineering of a complex organ: progress toward development of a tissue-engineered lung. Proc Am Thorac Soc, 2008. 5(6): p. 723-30.
84. Allman, A. J., et al., Xenogeneic extracellular matrix grafts elicit a TH2-restricted immune response. Transplantation, 2001. 71(11): p. 1631-40.
85. Allaire, E., et al., Cell-free arterial grafts: morphologic characteristics of aortic isografts, allografts, and xenografts in rats. J Vasc Surg, 1994. 19(3): p. 446-56.
86. Lu, H., et al., Autologous extracellular matrix scaffolds for tissue engineering. Biomaterials, 2011. 32(10): p. 2489-99.

What is claimed is:

1. A method comprising:
differentiating preadipocytes;
culturing the differentiated preadipocytes in media;
collecting the extracellular matrix proteins secreted by the preadipocytes;
filtering the extracellular matrix proteins; and
collecting retentate from said filtering.

2. The method of claim 1, wherein the retentate is a gel.

3. The method of claim 1, further comprising using the retentate as a scaffold.

4. The method of claim 1, wherein the retentate comprises extracellular matrix proteins larger than 100 kDa.

5. The method of claim 1, further comprising using the retentate to promote cell adhesion.

6. The method of claim 1, wherein collecting the extracellular matrix proteins secreted by the preadipocytes comprises collecting the media in which the preadipocytes are cultured.

7. The method of claim 1, wherein filtering the extracellular matrix proteins comprises using a centrifuge filter.

8. The method of claim 1 wherein the retentate comprises extracellular matrix proteins and growth factors.

9. The method of claim 1, further comprising culturing islet cells in the presence of the retentate.

10. The method of claim 1, wherein the media comprises at least one of rosiglitazone, proline, osteocalcin, hydroxyproline, oleic acid, palmitic acid, and linoleic acid.

11. The method of claim 1 wherein the preadipocytes are Simpson-Golabi-Behmel Syndrome preadipocytes.
